(19) United States
(12) Patent Application Publication
(10) Pub. No.: US 2005/0214325 A1
(43) Pub. Date: Sep. 29, 2005

(54) Compositions and methods to increase the effect of a neurotoxin treatment

(75) Inventor: Nathaniel E. David, San Francisco, CA (US)

Correspondence Address: WILSON SONSINI GOODRICH & ROSATI, 650 PAGE MILL ROAD, PALO ALTO, CA, 94304-1050, US

(73) Assignee: VVII NewCo 2003, Inc., Menlo Park, CA (US)

(21) Appl. No.: 10/810,391

(22) Filed: Mar. 26, 2004

(51) Int. Cl. A61K 39/08; A61K 38/54

(52) U.S. Cl. 424/239.1; 514/12

(57) ABSTRACT

The present invention discloses compositions and methods for enhancing the effect (e.g., duration) of a neurotoxin treatment. The compositions herein include neurotoxins and neuron growth inhibitors. Such compositions are administered locally to treat or prevent conditions, such as dermatological conditions, urological conditions, thyroid conditions, optical conditions, and neurological conditions.

### BACKGROUND OF THE INVENTION

Neurotoxins are often used for the treatment and prevention of various diseases as well as for cosmetic applications. A commonly used neurotoxin is botulinum toxin type A. Botulinum toxin type A is a member of a family of toxins that was first discovered by Professor Emile Pierre van Ermengem in 1895. The botulinum toxins were isolated and purified in the 1920s by Dr. Herman Sommer at the University of California, San Francisco. Botulinum toxin type A was separated out from the other types of botulinum toxins in the 1960's. By the 1970's, type A was found to be effective in treating neuronal disorders, such as those related to involuntary crossing of the eyes and related to neck and head spasms. Since then, other botulinum toxin types (e.g., botulinum toxin types B, C, D, E, F, and G) have also been isolated and have shown to be effective in the treatment of various conditions. Today, botulinum toxin type A is the most commonly used botulinum toxin and is approved for the treatment of brow wrinkle removal, and optical conditions, such as blepharospasm, strabismus, and Duane's syndrome. However, the use of neurotoxins, including botulinum toxin type A, may be risky and may cause severe side effects. Examples of side effects caused by botulinum toxin type A include, but are not limited to, flu like symptoms, weakness in the group of muscles being treated, difficulty swallowing, collapsed lung, etc.

Thus, it would be desirable to identify compositions and methods that increase the effect of a neurotoxin treatment (e.g., increase the duration of effect of a neurotoxin treatment), thereby reducing the amount of neurotoxin administered per application or the number of applications per treatment cycle. An additional benefit of such compositions and methods includes reducing the antigenicity to the neurotoxin.

### SUMMARY OF THE INVENTION

The present invention relates to compositions and methods that increase the efficacy of a neurotoxin treatment. Enhancing the efficacy of a neurotoxin treatment can take place, or example, by inhibiting or delaying neurojunction repair or by delaying, reducing, inhibiting or interfering with the process neuronal growth and/or axonal sprouting.

A neurojunction can be any junction with a neuron. In preferred embodiments, the neurojunction is a neuromuscular junction between a neuron and a muscle cell. In such junctions, neurotransmission is usually conducted by a neurotransmitter (e.g., Acetylcholine (ACh)). Repair and/or reconstruction of a neurojunction typically involve neuronal cell growth and/or axonal sprouting.

In some embodiments, the methods herein include administering locally to a target region of a mammal a neurotoxin and a neuron growth inhibitor. The neurotoxin is preferably a botulinum toxin selected from the group consisting of botulinum toxin types A, B, C, D, E, F, and G. More preferably, the neurotoxin is botulinum toxin is of type A. The neuron growth inhibitor may be any agent that inhibits neuronal cell growth and/or axonal sprouting. In preferred embodiments, the neuronal growth inhibitor is selected from the group consisting of a Trk receptor inhibitor, a Ras inhibitor, a Raf inhibitor, a Rap-1 inhibitor, a MEK inhibitor, an ERK inhibitor, a PKA inhibitor, a PKC inhibitor, a p53 inhibitor, a growth factor inhibitor, or an inhibitor of any activator or effector of any of the above. In preferred embodiments, the neuron growth inhibitor is a MEK inhibitor or a Raf inhibitor (e.g., a b-Raf inhibitor). A MEK inhibitor is preferably selected from the group consisting of PD98059, U0126, PD 184352, 2-Cholor-3-(N-succinimidyl)-1,4-naphthoquinone, PD 184352, ARRY-142886, tricyclic flavone, and 2-(2-amino-3-methoxyphenyl)-4-oxo-4H-[1]benzopyran.

A Raf inhibitor is preferably Rheb, BAY-43-9006 or a Raf kinase protein inhibitor (RKPI).

A neurotoxin can be administered prior to, simultaneous with, or after administration of a neuron growth inhibitor. In preferred embodiments, the neurotoxin is administered after the administration of the neuron growth inhibitor.

In preferred embodiment, both the neurotoxins and the neuron growth inhibitors are administered locally. Means for localized administration include any method known in the art, but preferably by topical, transdermal, subdermal, subcutaneous, or intramuscular administration.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a typical signal transduction pathway via MAPK.

### DETAILED DESCRIPTION OF THE INVENTION

1. Neurotransmission

The nervous system coordinates movements of the body and cellular activities. Most neurons achieve their effect by releasing chemicals, such as neurotransmitters. Neurotransmitters are released from the axon terminal of one neuron, and pass a junction known as the synapse, before reaching a receiving cell (a postsynaptic cell). A postsynaptic cell can be, for example, another neuron, a muscle cell, or a gland cell. Neurotransmitters at excitatory synapses depolarize a postsynaptic cell membrane.

A neurotransmitter that is commonly used throughout the body is Acetylcholine (ACh). Acetylcholine is known to activate two types of receptors, muscarinic and nicotinic receptors. The muscarinic receptors are found in all effector cells stimulated by the postganglionic neurons of the parasympathetic nervous system, as well as in those stimulated by the postganglionic cholinergic neurons of the sympathetic nervous system. The nicotinic receptors are found in the synapses between the preganglionic and postganglionic neurons of both the sympathetic and parasympathetic. The nicotinic receptors are also present in many membranes of skeletal muscle fibers at the neuromuscular junction.

Acetylcholine is released from cholinergic neurons when intracellular vesicles fuse with the presynaptic neuronal cell membrane. Vesicles are generally about 50 nm in diameter and contain about 10,000 molecules of ACh. Vesicle precursors are made in the endoplasmic reticulum (ER) and golgi of the neuronal soma and are transported down the axon to the terminal where the membrane pinches off to create new vesicles.

It is postulated that when ACh binds to its receptors on the postsynaptic cell membrane ligand-gated sodium channels opens up. These ligand-gated sodium channels allow an influx of Na⁺ ions, which in turn reduces the membrane potential of the postsynaptic cell to an excitatory postsynaptic potential (EPSP). If depolarization of the postsynaptic membrane reaches a particular threshold, an action potential is generated in the postsynaptic cell.

Defects in synaptic vesicle release and/or recycling can cause severe neurological and neuromuscular disorders. Such disorders include, but are not limited to, myasthenic syndromes such as Lambert-Eaton myasthenic syndrome (LEMS), Congenital myasthenic syndrome, botulism, and tetanus toxicity. Defects in synaptic vesicle release and/or recycling can be effectuated by neurotoxins, especially the neurotoxins. In particular, the present invention contemplates the administration of neurotoxins for the inhibition, delay, interference, or decrease of vesicle release and/or recycling.

2. Neurotoxins

The present invention relates to compositions and methods for improving neurotoxin treatment, e.g., by increasing the duration of the effect of a neurotoxin. Such compositions and methods are useful in the treatment and prevention of a condition that is treatable or preventable by a neurotoxin.

The term “neurotoxin” refers to any substance that inhibits neuronal function. Neurotoxins are often extremely toxic if taken or applied inappropriately. Neurotoxins can function, for example, against sodium channels (e.g., tetrodotoxin) or by blocking synaptic transmission (e.g., curare and bungarotoxin, botulinum toxin).

Examples of neurotoxins include, but are not limited to, curare, bungarotoxin, saxitoxin, tetrodotoxin, tetanus toxin, and botulinum toxins. Curare neurotoxins are alkaloids that are the active ingredients of arrow poisons used by South American Indians. Curare alkoids have muscle relaxant properties because they block motor end plate transmission, acting as competitive antagonists for acetylcholine. Bungarotoxin is a neurotoxic protein derived from the venom of an elapid snake known as bungarus multicinctus. Alpha-bungarotoxin blocks nicotinic acetylcholine receptors, while beta- and gamma-bungarotoxins act presynaptically causing acetylcholine release and depletion. Saxitoxin is a neurotoxin produced by the red tide dinoflagellates, Gonyaulax catenella and G. Tamarensis. Saxitoxin binds to sodium channels, thus blocking the passage of action potentials. This toxin was originally isolated from the clam, Saxidomus giganteus. Tetrodotoxin is a neurotoxin derived from the Japanese puffer fish. Tetrodotoxin also binds to sodium channel, and its activity somewhat resembles that of saxitoxin. Tetanus toxin is a neurotoxin caused by the anaerobic, spore-forming bacillus Clostridium tetani. Clostridium tetani usually enters the body through contaminated puncture wounds although it may also enter through burns, surgical wounds, cutaneous ulcers, injection sites etc. Tetanus toxicity is often accompanied with sustained muscular contraction caused by repetitive nerve stimulation. Botulinum neurotoxins are produced by the anaerobic, gram-positive bacterium Clostridium botulinum (referred to herein as C. botulinum). Botulinum toxins can cause neuroparalysis, or botulism, in mammals. There are at least seven known types of botulinum toxins: toxins A, B, C₁ (referred to herein as “C”), D, E, F, and G.

The molecular weight of each one of the above seven types of botulinum toxin is about 150 kD. When these botulinum toxins are released by C. bacterium, they are complexed with non-toxin proteins. For example, botulinum toxin type A complex can be produced by Clostridial bacterium as either a 900 kD, 500 kD, or a 300 kD form. Botulinum toxin type B and C are usually produced as a 500 kD complex. Botulinum toxin type D is usually produced as either a 300 kD or a 500 kD complex. Finally, botulinum toxin types E and F are usually produced as an ˜300 kD complexes.

These complexes of molecular weight greater than about 150 kD are believed to contain a non-toxin hemaglutinin protein and a non-toxin and non-toxic nonhemaglutinin protein. These two non-toxin proteins may act to provide stability against denaturation to the botulinum toxin molecule and protection against digestive acids when toxin is ingested. Additionally, it is possible that the larger botulinum toxin complexes may result in a slower rate of diffusion of the botulinum toxin away from a site of intramuscular injection of a botulinum toxin complex.

While each one of the botulinum neurotoxins has different properties and actions, there are some general structural and functional similarities among all seven botulinum toxins. For example, all seven toxins are synthesized as single-chain polypeptides with molecular weights of approximately 150-kD. These single-chain molecules are activated by proteolytic enzymes by nicking or cleaving. Once it is nicked or cleaved, the 150-kD single-chain molecule forms a dichain molecule consisting of a ˜100-kD heavy chain (H chain) and a 50-kD light chain (L chain) linked by a disulfide bond. The H chain is responsible for high-affinity docking of the neurotoxin to the presynaptic nerve terminal receptor, which enables the internalization of the neurotoxin into the cell. The L chain is a zinc-dependent endopeptidase that cleaves membrane proteins (e.g., SNAP-25 or VAMP) that are responsible for docking neurotransmitter vesicles (e.g., ACh vesicles) on the inner side of the nerve terminal membrane.

Thus, the molecular mechanism by which all of the botulism neurotoxins function can be summarized by the following three steps. In the first step, the neurotoxin binds to the presynaptic membrane of the target neuron through a specific interaction between the H chain and a cell surface receptor. The receptor for each type of botulinum neurotoxin and for tetanus neurotoxin is different. The carboxyl end segment of the H chain (Hc) appears to be important for targeting of the toxin to the cell surface. In the second step, the neurotoxin crosses the plasma membrane of the presynaptic cell. The neurotoxin enters the cell through receptor-mediated endocytosis. An endosome containing the neurotoxin is formed. Each endosomes contains a proton pump that decreases the pH inside the endosome. This reduced pH triggers a conformational change within the neurotoxin, which allows it to escape the endosome into the cytoplasm of the presynaptic cell. During the third phase, the disulfide bond joining the H and L chains is reduced. The L chain, which is a zinc (Zn++) endopeptidase, the selectively cleaves SNARE proteins. SNARE proteins, which include syntaxin, VAMP, and SNAP-25 are essential for recognition, docking, release and recycling of neurotransmitter-containing vesicles.

Each neurotoxin specifically cleaves a different amino acid bond of a SNARE protein. For example, the tetanus neurotoxin and the botulinum neurotoxin types B, D, F, and G degrade synaptobrevin (also known as “vesicle-associated membrane protein” or VAMP). Botulinum toxin type B cleaves VAMP at Gln76-Ph77. Botulinum toxin type D cleaves VAMP at Lys59-Leu-60. Botulinum toxin type F cleaves VAMP at Leu58-Lys59. And, botulinum toxin type G cleaves VAMP at a single Ala-Ala bond. VAMP is a synaptosomal membrane protein that is essential for vesicle release. Most of the VAMP present at the cytosolic surface of the synaptic vesicle is removed as a result of any one of the above cleaving events.

Similarly, botulinum neurotoxin types A and E block the release of ACh by cleaving a synaptosome-associated protein of molecular weight 25 kilodaltons, also known as SNAP-25. Botulinum toxin type A cleaves SNAP-25 at Gln197-Arg198, and botulinum toxin type E cleaves SNAP-25 at Arg180-Ile181. SNAP-25 is a plasma membrane protein that is located on the internal side of the plasma membrane of presynaptic nerve cells. SNAP-25 is integral to the vesicle release process. It is believed that the potency and duration of action of toxin type A derive, at least in part, from its action on SNAP-25. See Billante, C R., Muscle & Nerve, 26: 395-403 (2002).

Botulinum neurotoxin type C also cleaves SNAP-25. In addition, type C also cleaves the protein syntaxin. Syntaxin is a presynaptic membrane protein that is associated with calcium channels and SNAP-25. Botulinum neurotoxin type C is a zinc-endopeptidase that cleaves syntaxin isoform 1A at the Lys253-Ala254 peptide body and syntaxin isoform 1B at the Lys252-Ala253 peptide bond, only when they are inserted into a lipid bilayer. Syntaxin isoforms 2 and 3 are also cleaved by Botulinum neurotoxin type C. However, syntaxin isoform 4 is resistant to botulinum neurotoxin type C cleaving. See Schiavo G., J. Biol. Chem., 5: 270(18): 10566-70 (1995).

The cleavage of all of these proteins prevents fusion of the vesicles with the terminal nerve membrane. This, in turn, prevents the release of neurotransmitters (e.g., ACh) into the neuromuscular junction or synapse. It should be noted that while neurotoxins like botulinum toxin type A, prevent the release of ACh, they do not affect its synthesis or storage in the presynaptic neuron. Furthermore, they do not affect the conduction of electrical signals by such cells.

3. Neuron Growth Inhibitors

While the above neurotoxins denervate the presynaptic cells, evidence indicates that the presynaptic cell actually expands its endplate region in response to neurotoxins. For example, it has been shown that recovery from the effects of botulinum toxin type A is in part due to sprouting of new axons around toxin-blocked receptors to reestablish neuromuscular pathways. See Billante, C. R., et al., Muscle & Nerve, 26: 395-403 (2002). In particular, recovery from botulinum toxin type A has been found to have two distinct phases. In the first phase, recovery is due to neuron reinnervation by sprouting. In the second phase, neuron innervation is a result of unblocking of the original nerve terminals with retraction of nerve sprouting. Id.

Thus, the present invention also contemplates the inhibition of neurotransmission by administering one or more neurotoxins and one or more neuron growth inhibitors to a target region. The term “neuron growth inhibitor” as used herein refers to any substance that inhibits, interferes with, reduces, or decreases neuron and/or axonal growth (e.g., sprouting). Thus, a neuron growth inhibitor can be useful in increasing the efficacy of a neurotoxin by delay repair of a neurojunction, for example.

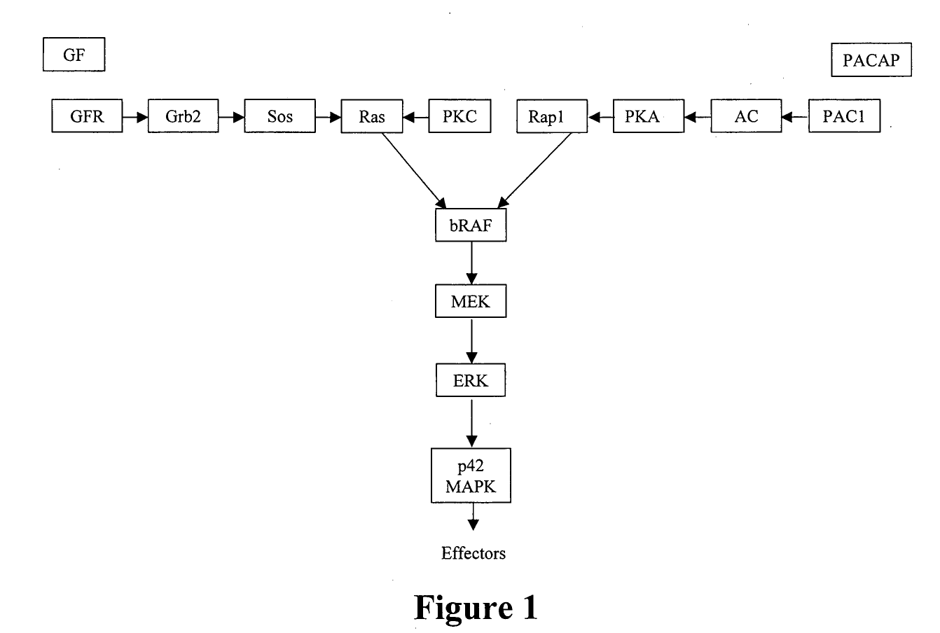

In preferred embodiments, a neuron growth inhibitor is any substance that interferes with the MAPK pathway or its activation of MEK/ERK. MAPK has been suggested to be involved in synaptic plasticity in post-mitotic cells of the central nervous system (CNS). For example, some studies suggest that MAPK is necessary for long-term facilitation of Aplysia sensory neuron-motor neuron synapses, associative conditioning in Hermissenda, and hippocampal long-term potentiation in rodents. See Adams, J. P., Neural Notes, Vol. 1, Issue 1 (1999). The MAPK cascade is regulated by a succession of kinases. A typical signal transduction pathway via MAPK is illustrated in FIG. 1. In FIG. 1, a growth factor (GF) (e.g., epidermal growth factor (EPG) or neuronal growth factors (NGF)) binds its growth factor receptor (GFR) on the cell surface. Growth factor receptors are generally tyrosine kinase (Trk) receptors.

There are three types of Trk receptors, each of which can be activated by one or more of the following four neurotrophis: NGR, brain-derived neurotrophic factor (BDNF), and neurotrophins 3 and 4 (NT3 and NT4). See Huang, E J., Annual Review of Biochemistry, Vol. 72, p. 609-642 (2003). Neurotrophin signaling through these receptors regulates, in part, cell survival, proliferation, and axon and dendrite growth and patterning. Id. Another type of receptor that function as signal transductors of neurotrophins is p75NTR. P75NTR is a member of the TNF receptr superfamily and is an effector of NF-kB.

In general, the binding of a growth factor to its Trk receptor causes that receptor to dimerize with an identical receptor. This dimerization initiates an autophosphorylation of tyrosine residues on the intracellular tail of the dimerized receptors. The phosphotyrosines that result from the autophosphorylation function as docking sites for signaling molecules such as Grb2 (an adaptor protein), SOS (a guanine nucleotide exchange factor) and Ras (a GTP binding protein). Other molecules that are activated by Trk receptors include Rap-1, and the Cdc-42-Rac-Rho family, P13K, and phospholipase-C-gamma.

In particular, the Grb2-SOS complex activates the small G-protein, Ras, by stimulating the exchange of guanosine diphosphate (GDP) for guanosine triphosphate (GTP). Other activators of Ras include, but are not limited to Phospholipase C and calmodulin (e.g., in response to calcium influx). Rit and Rin and two homologous Ras-like proteins that are plasma membrane-localized. Rin binds calmodulin through a C-terminal binding motif. It has been suggested that Rit and Rin define a novel subfamily of Ras-related proteins, perhaps using a new mechanism of membrane association, and that Rin may be involved in calcium-mediated signaling within neurons. See Lee, CHJ, et al., The Journal of Neuroscience, Vol. 16, No. 21, pp. 6784-6794 (1996).

Activation of Ras is associated with the promotion of cell proliferation (mediation of growth factors), cell differentiation (e.g., PC12 cells), and differentiation of cell functions (mediate calcium signaling). Ras is a notable member of the large family of GTPases, proteins that bind and hydrolyze GTP. The Ras superfamily, which includes approximately 50 different members, can be divided into subfamilies according to function and sequence. One subfamily is associated with cell growth and differentiation includes the following members: H-Ras, N-Ras, K-Ras, TC-21, Rap-1, Rap-2, R-Ras, Ral-A, Ral-B. Another Ras superfamily associated with cytoskeleton structuring includes the following members: Rho-A, Rho-B, Rho-G, Rho-E, CDC-42, Rac-1, and Rac-2. A third Ras superfamily associated with vesicle sorting includes the following members: Rab, Arf, and Ran.

Effectors of Ras include but are not limited to phosphatidylinositol-3′-kinase (PI3K), Raf, and Ral. Over-expression of PI3K is associated with enlarged cell somata and axon width. Id. It is also a known activator of Atk, a serine/threonine kinase that is essential for growth dependent survival of neurons. See Markus A., Neuron, Vol. 35: 65-76 (2002). Over-expression of Atk is associated with an in increase in the number of axon branches as well as enlargement of the cell somata. Id.

Ras activates MEK and ERK by a central three-tiered core signaling module, which comprises of an apical MAPK kinase kinase (MAP3K), a MAPK kinase (MEK or MKK), and a downstream MAPK. MEK in turn phosphorylates and activates extracellular-signal-regulated kinase (ERK).

The most common MAP3K is Raf. The exchange of guanosine diphosphate (GDP) for guanosine triphosphate (GTP) by Ras elicits a conformational change, which enables it to bind and activate Raf. The Raf kinase family is a serine/threonine protein kinase which catalyzes hydroxyl groups on specific serine and threonine residues. Interaction of Ras with Raf is thought to be necessary but not sufficient to activate Raf. Mammals possess 3 Raf proteins, ranging from 70 to 100 kDa in size. These Raf isomers are known as: a-Raf, b-Raf, and c-Raf or Raf-1. While Raf-1 is ubiquitously distributed throughout the body, a-Raf is found abundantly in urogenital tissue and b-Raf is found predominantly in neuronal tissue.

Generally, Ras recruits Raf (e.g., b-Raf or Raf-1) from the cytosol to the cell membrane, where Raf is activated. Raf activation is thought to involve a multi-step process that includes the dephosphorylation of inhibitory sites by protein phosphatase 2A (PP2A) and the phosphorylation of activating sites by PAK (p21^{rac/cdc42}-activated kinase), Src-family and some unknown kinases.

B-Raf kinases are associated with extracellular signaling that suppress apoptosis and regulate cell differentiation. Additional activators of b-Raf include, PKA, PKB, PKC, KSR, Pak, and 14-3-3. While PKA inhibit Raf-1 catalytic activity in most cells, it potentiates nerve growth factor-stimulated PC12 cell differentiation, which is a b-Raf mediated process. This potentiation rather than inhibition of PC12 cell differentiation is thought to be the result of the N-terminal regulatory domain of PKA. It is believed that this domain interferes with the ability of PKA to modulate b-Raf catalytic activity and provides resistance of b-Raf-dependent processes to PKA inhibition.

Rheb (Ras homolog enriched in brain) is a new class of G-proteins and is a member of the Ras superfamily and an immediate member of the Rap/Ral subfamily. Rheb, like Ras and Rap1, binds b-Raf kinase, but in contrast to Ras and Rap 1, Rheb inhibits b-Raf kinase activity and prevents b-Raf-dependent activation of the transcription factor Elk-1. Rheb homologs can be define based on their overall sequence similarity, high conservation of their effector domain sequence, presence of a unique arginine in their G1 box, and presence of a conserved CAAX farnesylation motif.

MEK is a unique kinase in that it phosphorylates MAPK on both threonine and tyrosine residues. MEK is the only known activator of MAPK, and MAPK is the only known target of MEK. Activated ERK has many substrates in the cytosol (e.g. cytoskeletal proteins, such as MAP and Tau, nuclear transcription factors such as Elk, Myc, Fos, and Jun, signaling molecules such as cytosolic phospholipase A2, and other kinases such as RSK. Id. Inhibition of MEK/MAPK and p53 pathways has been associated with nerve growth inhibition. See Pumiglia, K. M., Proc. Natl. Acad. Sci. USA, Vol. 94, 448-452 (January 1997); see also Adams, J. P., Neural Notes, Vol. V, Issue 1, 14-16 (1999); see also Mazzoni, I. E., J. Neurosci. 19(22): 9716-27 (November 1999).

In neuronal cell lines such as PC 12, NGF and EGF have been shown to use the same Raf/MEK/ERK pathway to cause PC12 proliferation and differentiation. However, another pathway exists to induce neuronal endplate growth. This pathway is activated by the pituitary adenylate cyclase-activating polypeptide (PACAP). See Vaudry, D., Science, Vol 296: 1648-49 (2002). PACAP has been found to cause robust neurite outgrowth by activating ERK. PACAP signaling is believed to be independent of Ras. PACAP is thought to activate adenylate cyclase (AC), which increases intracellular cAMP. cAMP, in turn, activates ERK through PKA. However, inhibition of PKA with H89 does not seem to block activation of ERK. Id. This suggests that cAMP may activate ERK through the Raf/MEK/ERK pathway, e.g., via Rap-1 or another effector.

Thus, the present invention contemplates the use of a neuron growth inhibitor in combination with a neurotoxin for the treatment and/or prevention of various conditions. In preferred embodiments, a neuron growth inhibitors is selected from the group consisting of a Trk receptor inhibitor, a Ras inhibitor, a Raf kinase inhibitor, a Rap-1 inhibitor, a PKA inhibitor, a p53 inhibitor, a MEK inhibitor, an ERK inhibitor, a NF-kB inhibitor, am inhibitor of a growth factor (e.g., NGF), or an inhibitor of an isozyme, derivative, splicing variant, activator or effector (target) of any of the above (e.g., Ras, Raf, Rap-1, etc.).

Examples of MEK inhibitors include but are not limited to SL327, PD98059 (CalBiochem Cat. No. 513000), U0126 (CalBiochem Cat. No. 662005), PD 184352 (see Delaney, A. M., Molec. Cell Biol., Vol. 22, No. 21, p. 7593-7602 (2002); 2-Cholor-3-(N-succinimidyl)-1,4-naphthoquinone (CalBiochem Cat. No. 444938), ARRY-142886 (AstraZeneca), tricyclic flavone, and 2-(2-amino-3-methoxyphenyl)-4-oxo-4H-[1]benzopyran.

Examples of Ras inhibitors include, but are not limited to, N17Ras and farnesyltransferase inhibitors (FTIs), such as FTI-277 and nontoxic farnesylcysteine analogue farnesylthiosalicylic acid (FTS), which dislodges all Ras isoforms from the membrane. See Kloog, Y., Mol. Med. Today, 6(10): 398-402 (2000); see also Aletsee, C., JARO, (O₂) 377-378 (2001).

Examples of P13-K inhibitors include, but are not limited to, LY294002.

Examples of compounds that inhibit the Raf-Ras interaction include, but are not limited to, those short peptides disclosed in Zeng, J., Protein Engineering, Vol. 14, No. 1, 39-45, (2001) and MCP1 and its derivatives, 53 and 110 (see Kato-Stankiewicz, J., Proc. Natl. Acad. Sci. USA., 99 (22): 14398-14403 (2002)).

Examples of b-Raf inhibitors include, but are not limited to, bis-aryl ureas, such as, e.g., BAY-43-9006, which inhibit b-Raf (see Wilhelm S., Current Pharmaceutical Design, Vol. 8, No. (2002)), Rheb (Ras homolog enriched in brain), which inhibits b-Raf, and RKIP (Raf kinase inhibitor protein).

An example of a PKA inhibitor is H-89.

Examples of PKC inhibitors include competitive inhibitors for the PKC ATP-binding site, including staurosporine and its bisindolylmaleimide derivitives, Ro-31-7549, Ro-31-8220, Ro-31-8425, Ro-32-0432 and Sangivamycin; drugs which interact with the PKC's regulatory domain by competing at the binding sites of diacylglycerol and phorbol esters, such as calphostin C, Safingol, D-erythro-Sphingosine; drugs which target the catalytic domain of PKC, such as chelerythrine chloride, and Melittin; drugs which inhibit PKC by covalently binding to PKC upon exposure to UV lights, such as dequalinium chloride; drugs which specifically inhibit Ca-dependent PKC such as Go6976, Go6983, Go7874 and other homologs, polymyxin B sulfate; drugs comprising competitive peptides derived from PKC sequence; and other PKC inhibitors such as cardiotoxins, ellagic acid, HBDDE, 1-O-Hexadecyl-2-O-methyl-rac-glycerol, Hypercin, K-252, NGIC-I, Phloretin, piceatannol, Tamoxifen citrate. Additional inhibitors shown to be effective include: 542 (+−)-1-(5-Isoquinolinesulfonyl)-2-methylpiperazine dihydrochloride; IC50=6.0 μM; 543 1-(5-Isoquinolinesulfonyl)piperazine; IC50=6.0 μM; 609 (+/−)-Palmitoylcarnitine chloride; 621 10-[3-(1-piperazinyl)propyl]-2-trifluoromethylphenothiazine dimaleate; 632 (+/−)-Stearoylcarnitine chloride. Alternative pharmacologically acceptable inhibitors effective in the disclosed methods are readily screened from the wide variety of PKC inhibitors known in the art (e.g Goekjian et al., Expert Opin. Investig. Drugs, 10, 2117-40 (2001); see also Battaini, Pharmacolog. Res., 44, 353-61 (2001). See U.S. Pat. No. 6,664,266, assigned to Children's Medical Center Corporation, incorporated herein by reference for all purposes.

An example of a Rap-1 inhibitor is SB203580.

In some embodiments, the invention herein utilizes a MEK inhibitor such as PD98059 to inhibit or delay neurojunction repair. In some embodiments, the invention herein utilizes a Raf kinase inhibitor, or more preferably, a b-Raf kinase inhibitor (e.g., Rheb or BAY-43-9006) to inhibit or delay neurojunction repair.

In addition, a neuron growth inhibitor of the present invention may also be an antisense, an antibody, a small or large organic or inorganic molecule, or any other compound that reduces or arrests the growth of nerve cells.

The term “antisense oligonucleotide” or “antisense,” as used herein, describes composition that include a nucleic acid sequence which specifically hybridizes under physiological conditions to a target DNA or RNA thereby inhibiting its transcription and/or translation. Antisense oligonucleotides include siRNA. (See Liang Y, et al., Clin Cancer Res. 2003: 19(16 suppl): 77. Abstract A111.) Antisense oligonucleotides can comprise of oligoribonucleotide, oligodeoxyribonucleotide, modified oligoribonucleotide, or modified oligodeoxyribonucleotide, and any derivatives, variants, fragments, and/or mimetics thereof. Antisense oligonucleotides can be naturally occurring or synthetic.

Those skilled in the art will recognize that the exact length of the antisense oligonucleotide and its degree of complementarity with its target will depend upon the specific target selected, including the sequence of the target and the particular bases which comprise that sequence. It is preferred that the antisense oligonucleotide be constructed and arranged so as to bind selectively with the target under physiological conditions, i.e., to hybridize substantially more to the target sequence than to any other sequence in the target cell under physiological conditions.

Thus in preferred embodiments, an antisense oligonucleotide can specifically hybridize with DNA or RNA of b-Raf, Ras, Rap-1, MEK, PKA, PI3-K, Akt, p53, ERK, a growth factor, (e.g., NGF), any elements that are upstream or downstream in the MAPK/MEK/ERK pathway or p53 pathway, and/or any derivative, variant, mimetic, or fragment of any of the above.

The term “antibody” or “antibodies,” as used herein, refers to any immunoglobulin that binds specifically to an antigenic determinant. Examples of antibodies include, but are not limited to, monoclonal antibodies, polyclonal antibodies, humanized antibodies, chimeric antibodies, single chain antibodies, Fab fragments, F(ab′)₂ fragments, fragments produced by FAb expression library, anti-idiotypic (anti-Id) antibodies, epitope-binding fragments of any of the above. Antibodies can be any immunoglobulin (e.g., IgG, IgM, IgA, IgE, IgD, etc.) obtained from any source (e.g., humans, rodents, non-human primates, lagomorphs, caprines, bovines, equines, ovines, etc.). In some embodiments, an antibody is directed against a species (e.g., anti-mouse, anti-human, etc.).

In preferred embodiments, a neuron growth inhibitor can be an antibody, more preferably a monoclonal antibody, or more preferably a chimeric or humanized antibody. Such antibody can preferably specifically bind to any one of the proteins that enhances neuronal growth or collateral axonal sprouts.

For example, the present invention contemplates a neuronal growth inhibitor that is an antibody that can specifically bind to Raf, Ras, Raf, MEK, PI3-K, Akt, p53, ERK, a growth factor, (e.g., NGF), any elements that are upstream or downstream in the MAPK/MEK/ERK pathway or p53 pathway, and/or any derivative, variant, mimetic, or fragment of any of the above. In preferred embodiments, a neuronal growth inhibitor is a monoclonal antibody that can specifically bind to MEK or ERK or Raf or b-Raf.

III. Indications For Treatment

The present invention contemplates the use of at least one neurotoxin and/or at least one neuron growth inhibitor for the treatment and prevention of a disease. Such a disease can include by way of example, any neurological, neuromuscular, urological, dermatological, and optical condition. Such conditions may further be characterized by involuntary muscle spasms, chronic pain, and/or aging skin.

In some embodiments, the present invention contemplates the use of at least one neurotoxin and/or at least one neuron growth inhibitor for the treatment and prevention of any condition for which a neurotoxin is used as a therapeutic agent. For example, today, botulinum toxin type A is approved for use for brow wrinkle removal, blepharospasm, strabismus, and Duane's syndrome. Blepharospasm is a condition associated with uncontrollable twitching of an eyelid that can be benign and/or related to stress, sleep deprivation, or the use of stimulants. Strabismus is an eye disorder wherein the optic axes cannot be directed to the same object. Duane's syndrome is a hereditary congenital syndrome in which the affected eye shows limitation or absence of abduction, restriction of adduction, retraction of the globe on adduction, narrowing of the palpebral fissure on adduction and widening on adduction, or deficient convergence.

Thus, the present invention contemplates the use of at least one neurotoxin and/or at least one neuron growth inhibitor for the treatment or prevention of dermatological and optical conditions such as brow wrinkle removal, blepharospasm, strabismus, and Duane's syndrome. Administration of the neurotoxin and/or the neuron growth inhibitor are preferably made locally (e.g., topically, subdermally, intramuscularly, or subcutaneously). In a combination treatment, the neurotoxin may be administered prior to, simultaneous with, or after the administration of the neuron growth inhibitor. In preferred embodiments, the neurotoxin is administered prior to the administration of the neuron growth inhibitor.

Neurotoxins may also be used for the treatment or prevention of localized dystonia. Examples of localized dystonia include, but are not limited to, cervical dystonia, embouchure dystonia, oromandibular dystonia, spasmodic dystonia, and writer's cramp. Cervical dystonia, also known as spasmodic torticollis, is a localized dystonia that is characterized by neck muscles contracting involuntarily. This may result in abnormal movements and posture of the head and neck. Embouchure dystonia is a term used to describe a type of dystonia that affects brass and woodwind players. Embouchure dystonia causes excessive twitching of the lips and may also cause forceful contractions of the jaw and tongue. Thus, patients suffering from oromandibular dystonia may experience difficulty in opening and closing their mouths as well as chewing and speaking. Spasmodic dystonia involves involuntary “spasms” of the vocal cords which may cause interruptions in speech and changes in voice quality. Furthermore, writer's cramp is a form of a localized dystonia, which is task specific and usually affects the hand and/or the arm.

Thus, in some embodiments, the present invention contemplates administration of at least one neurotoxin and/or at least one neuron growth inhibitor for the treatment of a localized dystonia. For example, a dystonia such as cervical dystonia, embouchure dystonia, oromandibular dystonia, spasmodic dystonia, and writer's cramp dystonia may be treated by administering locally to a target region at least one neurotoxin and at least one neuron growth inhibitor. Preferably, a neurotoxin is administered prior to the administration of the neuron growth inhibitor.

Additional indications that may be treatable or preventable by the compositions and methods herein are neurological disorders. Such neurological disorders include, but are not limited to, migraine headache, chronic pain (e.g., chronic low back pain), chronic muscle pain (e.g., fibromyalgia), stroke, traumatic brain injury, localized pain (e.g., vulvodynia), cerebral palsy, meige syndrome, hyperhydrosis, tremor, achalasia, secondary and inherent dystonias, Parkinson's disease, spinal cord injury, multiple sclerosis, and spasm reflex.

The compositions and methods herein may be especially useful in the treatment and prevention of urological conditions. Examples of urological conditions include, but are not limited to, pelvic pain (e.g., interstitial cystitis, endometriosis, prostatodynia, urethral instability syndromes), pelvic myofiscial elements (e.g., levator sphincter, dysmenorrhea, anal fistula, hemorrhoid), urinary incontinence (e.g., unstable bladder, unstable sphincter), prostate disorders (e.g., prostatic hyperplasia, benign prostatic hyperplasia, prostatic enlargement, BPH prostatitis, prostate cancer), recurrent infection (secondary to sphincter spasticity), and urinary retention (secondary to spastic sphincter, hypertrophied bladder neck) and bladder dysfunction.

In some embodiments, the compositions and methods herein may be used to treat and prevent skin condition and/or enhance wound healing. Exemplary skin conditions include eczema, psoriasis, dermatitis, melonoma, pityriasis, such as pitiyriasis rosea, pityriasis rosacea and pityriasis rubra, and other cutaneous cell-proliferative disorders. Skin wounds include, for example, facial or bodily lacerations, whether elective (e.g., surgically introduced incisions) or non-elective (e.g., lacerations caused by car accident).

In some embodiments, the compositions and methods herein may be used to treat or prevent injury to the muscle. Examples of muscle injuries include, but are not limited to, contusions (bruises), lacerations, ischemia, strains, and complete ruptures.

In some embodiments, the compositions and methods herein may be used to treat thyroid disorder such as hyperthyroidism, hypothyroidism, Graves' disease, goiter, thyroiditis, cancer, and all other conditions that may result in hypothyroidism or hyperthyroidism.

In some embodiments, the compositions and methods herein can be used to suppress or reduce snoring noises.

IV. Pharmaceutical Compositions, Dosages, and Methods For Administering.

1. Pharmaceutical Compositions

Those of ordinary skill in the art will know, or can readily ascertain, how to obtain the neurotoxins of the invention, including the botulinum and tetanus toxins, in a pharmaceutically safe form. Such form is preferably nonteratogenic and does not induce a detectable immune response to the toxin antigen. For most of the neurotoxins of the invention, pharmaceutical safety will be dose-dependent such that relatively low dosages of toxin will be “safe” as compared to dosages which are known to be sufficient to produce disease.

Preferably, the neurotoxins and/or neuron growth inhibitors of the invention will be administered as a composition in a pharmaceutically acceptable carrier. To that end, presynaptic neurotoxin compositions and/or neuron growth inhibitors are prepared for administration by mixing a toxin the desired degree of purity with physiologically acceptable sterile carriers. Such carriers will be nontoxic to recipients at the dosages and concentrations employed. Ordinarily, the preparation of such compositions entails combining the neurotoxin with buffers, antioxidants such as ascorbic acid, low molecular weight (less than about 10 residues) polypeptides, proteins, amino acids, carbohydrates including glucose or dextrins, chelating agents such as EDTA, glutathione and other stabilizers and excipients. Such compositions may also be lyophilized and will be pharmaceutically acceptable; i.e., suitably prepared and approved for use in the desired application.

A pharmaceutical composition of the present invention may be formulated to be suitable for application in a variety of manners, for example, in a cream for topical application to the skin (e.g., for alopecia), in a wash, in a douche, in a powder for chaffing (e.g., for dermatitis), in a liquid, in a dry formulation (e.g., as a bath salt or bath powder), and the like. Other formulations will be readily apparent to one skilled in the art. In preferred embodiments, the compositions herein are preferably formulated for local administration. Preferably, the compositions are formulated for topical, subcutaneous, intramuscular, or transdermal administration.

For transdermal and topical administration, the neurotoxins and/or neuron growth inhibitors will preferably be formulated to enhance penetration to and across the stratum corneum of the skin. Those of ordinary skill in the art will be familiar with, or can readily ascertain the identity of, excipients and additives, which will facilitate drug delivery (particularly of peptides) across skin. For review in this respect, reference may be made to “Novel Drug Delivery Systems”, Chien, ed. (Marcel Dekker, 1992), the disclosure of which is incorporated herein by this reference to illustrate the state of knowledge in the art concerning drug delivery to and across the stratum corneum of skin.

When formulated as an ointment, the active ingredient (e.g., the neurotoxins and/or neuron growth inhibitors) can be employed, for example, with either paraffinic or a water miscible ointment base. Alternatively, the active ingredients can be formulated in a cream with an oil-in-water cream base. If desired, the aqueous phase of the cream base can include, for example at least 30% w/w of a polyhydric alcohol such as propylene glycol, butane-1,3-diol, mannitol, sorbitol, glycerol, polyethylene glycol and mixtures thereof.

A topical formulation can desirably include a compound that enhances absorption or penetration of the active ingredient through the skin or other affected areas. Examples of such dermal penetration enhancers include dimethylsulfoxide and related analogs. Topical formulation may further include, for example, antioxidants (e.g., vitamin E); buffering agents; lubricants (e.g., synthetic or natural beeswax); sunscreens (e.g., para-aminobenzoic acid); and other cosmetic agents (e.g., coloring agents, fragrances, oils, essential oils, moisturizers or drying agents). Thickening agents (e.g., polyvinylpyrrolidone, polyethylene glycol or carboxymethylcellulose) may also be added to the compositions.

The carriers utilized in the pharmaceutical compositions of the present invention may be solid-based dry materials for use in powdered formulations or may be liquid or gel-based materials for use in liquid or gel formulations. The specific formulations depend, in part, upon the routes or modes of administration.

Typical carriers for dry formulations (e.g., bath salts) include, but are not limited to, trehalose, malto-dextrin, rice flour, micro-crystalline cellulose (MCC), magnesium sterate, inositol, fructo-oligosaccharides FOS, gluco-oligosaccharides (GOS), dextrose, sucrose, talc, and the like carriers. Where the composition is dry and includes evaporated oils that produce a tendency for the composition to cake (i.e., adherence of the component spores, salts, powders and oils), it is preferable to include dry fillers which both distribute the components and prevent caking. Exemplary anti-caking agents include MCC, talc, diatomaceous earth, amorphous silica and the like, typically added in an concentration of from approximately 1% to 95% by-weight.

Suitable liquid or gel-based carriers are well-known in the art (e.g., water, physiological salt solutions, urea, methanol, ethanol, propanol, butanol, ethylene glycol and propylene glycol, and the like). Preferably, water-based carriers are approximately neutral pH.

Additional suitable carriers include aqueous and oleaginous carries such as, for example, white petrolatum, isopropyl myristate, lanolin or lanolin alcohols, mineral oil, fragrant or essential oil, nasturtium extract oil, sorbitan mono-oleate, propylene glycol, cetylstearyl alcohol (together or in various combinations), hydroxypropyl cellulose (MW=100,000 to 1,000,000), detergents (e.g., polyoxyl stearate or sodium lauryl sulfate) and mixed with water to form a lotion, gel, cream or semi-solid composition. Other suitable carriers comprise water-in-oil or oil-in-water emulsions and mixtures of emulsifiers and emollients with solvents such as sucrose stearate, sucrose cocoate, sucrose distearate, mineral oil, propylene glycol, 2-ethyl-1,3-hexanediol, polyoxypropylene-15-stearyl ether and water. For example, emulsions containing water, glycerol stearate, glycerin, mineral oil, synthetic spermaceti, cetyl alcohol, butylparaben, propylparaben and methylparaben are commercially available. Preservatives may also be included in the carrier including methylparaben, propylparaben, benzyl alcohol and ethylene diamine tetraacetate salts. Well-known flavorings and/or colorants may also be included in the carrier. The composition may also include a plasticizer such as glycerol or polyethylene glycol (MW 400 to 20,000). The composition of the carrier can be varied so long as it does not interfere significantly with the pharmacological activity of the active ingredient (botulinum toxin type A).

2. Dosages

When administering a neurotoxin (especially botulinum toxin type A) and/or a neuron growth inhibitor, small dosages should be applied. Generally, the dose of the neurotoxin and/or neuron growth inhibitor to be administered will vary depending on the age of the host being treated, sex and weight of the host, condition being treated, severity of such condition, location of the condition, and potency of the neurotoxin.

Toxin potency is expressed as a multiple of the LD₅₀ value for a reference mammal, usually a mouse. Where a mouse is the reference mammal, one “unit” of toxin is the amount of toxin that kills 50% of a group of mice that were disease-free prior to inoculation with the toxin. For example, commercially available botulinum toxin A typically has a potency such that one nanogram contains about 40 mouse units. It should also be noted that each neurotoxin, neurotoxin type, and/or neuron growth inhibitor may have its own LD₅₀ and that the LD₅₀ may vary depending on the animal species. The potency in humans of the botulinum toxin type A product supplied by Allergan, Inc. is believed to be about LD₅₀=2,730 units. Furthermore, it has been shown that botulinum toxin type A is 500 times more potent, as measured by the rate of paralysis produced in the rat, than is botulinum toxin type B.

Assuming a potency which is substantially equivalent to LD₅₀=2,730 units, the neurotoxin can be administered in a dose of up to about 2000 units, although individual dosages will be smaller. For example, a single application in a treatment cycle can include 0.25-50 units of a neurotoxin, more preferably 0.5-25 units of a neurotoxin, more preferably 1 to 10 units of a neurotoxin, more preferably 1.25-5 units of a neurotoxin, or more preferably 1.25-2.5 units of a neurotoxin. The present invention also contemplates administering smaller doses of a neurotoxin (especially in combination treatments). Such doses may be less than 5 units of a neurotoxin per application, less than 2 units of a neurotoxin per application, less than 1 units of a neurotoxin per application, or less than 0.5 units of a neurotoxin per application.

The above dosages for neurotoxins may be administered once or at recurring intervals or on an as need basis. For example, the above dosages may be administered once a day, more preferably about once a week, more preferably about once a month, more preferably about every 3 months, more preferably about every 6 months, or more preferably about every 9 months. Greater time intervals are also contemplated by the present invention. The dosage may also be adjusted upward or downward depending additional agents administered (e.g., a neuron growth inhibitor), the condition and severity of condition being treated, and the sex, age and specie of mammal being treated. Preferably, the lowest therapeutically effective dosage will be administered. In the initial treatment, a low dosage may be administered at a target site to determine the patient's sensitivity to, and tolerance of, the neurotoxin. Additional injections of the same or different dosages will be administered as necessary.

Thus, an effective amount of a neurotoxin is a dosage sufficient to delay, decrease, interfere, or inhibit neuronal transmission for at least one day, more preferably for at least one week, more preferably for at least one month, more preferably for at least 3 months, more preferably for at least 6 months, more preferably for at least 9 months, or more preferably for at least 1 year.

In any of the embodiments herein, a neuron growth inhibitor may be administered in addition to the neurotoxin. A combination treatment of a neuron growth inhibitor and a neurotoxin involves administering both an effective amount of at least one neurotoxin and an effective amount of at least one neuron growth inhibitor. When administering a combination treatment, the effective amount of either or both the neurotoxin and/or the neuron growth inhibitor may be less than in a single drug therapy due to the synergistic effect of both agents. A combination treatment of a neurotoxin and a neuron growth inhibitor can include administration of a neurotoxin prior to, contemporaneous with, or post administration of a neuron growth inhibitor. For example, a neuron growth inhibitor may be administered simultaneous to, immediately subsequent to, approximately 5 minutes subsequent to, about an hour subsequent to, about 2 hours subsequent to, about 6 hours subsequent to, about a day subsequent to, about 2 days subsequent to, about a week subsequent to, about 2 weeks subsequent to, about a month subsequent to, about 3 months subsequent to, or about 6 months, subsequent to a neurotoxin treatment. In preferred embodiments, the neuron growth inhibitor is administered simultaneous to or immediately following the administration of a neurotoxin.

In some embodiments, an effective amount of a neuron growth inhibitor is the dosage sufficient to delay, decrease, interfere, and/or inhibit neuronal and/or axonal growth (e.g., sprouting) for at least one day, more preferably for at least one week, more preferably for at least one month, more preferably for at least 3 months, more preferably for at least 6 months, more preferably for at least 9 months, or more preferably for at least 1 year.

In some embodiments, an effective amount of a neuron growth inhibitor is the dosage sufficient to delay, decrease, interfere, and/or inhibit neurotransmission for at least one day, more preferably for at least one week, more preferably for at least one month, more preferably for at least 3 months, more preferably for at least 6 months, more preferably for at least 9 months, or more preferably for at least 1 year.

Dosing of either or both the neurotoxin and/or neuron growth inhibitor can be single dosage or cumulative (serial dosing), and can be readily determined by one skilled in the art. For serial dosing (i.e., one dose per day, more preferably one dose per week, more preferably one dose per month, more preferably one time per every six months, more preferably one time per every eight months, or more preferably one time per every one year), a dosage schedule can be readily determined by one skilled in the art based on, e.g., patient size, condition to be treated, severity of the condition, neurotoxin selected, and other variables.

One suggested course of treatment and/or prevention involves the use of a neurotoxin (e.g., botulinum toxin type A) and a neuron growth inhibitor (e.g., a MEK inhibitor). The neurotoxin is administered at about 40 units every three days up to the LD₅₀ for the neurotoxin. More preferably, the neurotoxin is administered at about 20 units every three days up to the LD₅₀ for the neurotoxin. More preferably, the neurotoxin is administered at about 10 units every three days up to the LD₅₀ for the neurotoxin.

In some embodiments, a neuron growth inhibitor may be administered in addition to (or in substitution to) the neurotoxin. A neuron growth inhibitor may be administered at a dosage rate of about 0.01 milligrams/kg per day to 2000 milligrams/kg per day, more preferably at a dosage rate of about 0.1 milligrams/kg per day to 1000 milligrams/kg per day, more preferably at a dosage rate of about 1 milligrams/kg per day to 750 milligrams/kg per day, more preferably at a dosage rate of about 5 milligrams/kg per day to 500 milligrams/kg per day, more preferably at a dosage rate of about 10 milligrams/kg per day to 250 milligrams/kg per day, more preferably at a dosage rate of about 25 milligrams/kg per day to 100 milligrams/kg per day, or more preferably at a dosage rate of about 30 milligrams/kg per day to 75 milligrams/kg per day. The dosage rate can change depending on the length of time between each application (e.g., 1 milligrams/kg per day is equivalent to about 7 milligrams/kg per week.) and the type of neurotoxin administered in conjunction with the neuron growth inhibitor.

The neuron growth inhibitor may be administered prior to, simultaneous with, or subsequent to the administration the neurotoxin. In preferred embodiments, the neuron growth inhibitor is administered subsequent to the administration the neurotoxin. For example, a neuron growth inhibitor may be administered 1/2 hour subsequent to the administration of a neurotoxin, more preferably 1 hour subsequent to the administration of a neurotoxin, more preferably 6 hours subsequent to the administration of a neurotoxin, more preferably 12 hours subsequent to the administration of a neurotoxin, more preferably 1 day subsequent to the administration of a neurotoxin, or more preferably 1 week subsequent to the administration of a neurotoxin.

3. Methods For Administration

The present invention contemplates the administration of a neurotoxin and a neuron growth inhibitor to treat and/or prevent various conditions. Predisposition to a condition may be determined prior to administration of the compositions herein according to conventional clinical standards, such as a prior or contemporaneous diagnosis or family history of the disease. Thus, a person diagnosed with a predisposition to a condition (especially one that is known to be treatable by a neurotoxin) may be administered a neurotoxin and a neuron growth inhibitor to prevent such condition.

For many indications, (especially those having a localized effect e.g., localized dystonia, psoriasis, wrinkles, etc.) subcutaneous, subdermal or intramuscular injections at the target site will be the most efficacious route of administration. Preferably, the injection will be provided to the subcutaneous or subdermal region beneath or into a target region (e.g., muscle effected by dystonia, or wrinkles) by inserting the needle below or into the target area. However, where a target region is too large or otherwise not susceptible to this approach, the compositions herein may be administered by transdermal or topical routes one or more target sites. However, it is expected that these latter routes will be less efficacious than subcutaneous, subdermal or intramuscular injections and may, therefore, be best used for subacute manifestations.

The injections will be repeated as necessary. As a general guideline, it has been observed that, after administration of a neurotoxin (e.g., botulinum toxin type A) into or near a target region in adult human skin according to the method of the invention, the treated region has remained paralyzed (e.g., neurotransmission has been inactivated) for periods of at least 2 months. Botulinum toxin type A in particular is expected to be most effective when administered according to the methods herein soon after the appearance of any indication of a condition. Depending on the course of therapy applied (i.e., with respect to dosage, frequency of treatment and sensitivity of individual patients to treatment), the method of the invention can be expected to be effective in mitigating the condition (e.g., reducing wrinkles or other alleviating pain), inducing remission of the condition, and in controlling symptoms associated with the condition (e.g., scaling of lesions and/or pain).

The neurotoxins and neuron growth inhibitors of the present invention are preferably administered locally. Local administration can be made, for example, by topical, subcutaneous, transdermal, subdermal or intramuscular administration.

In some embodiments, the methods of the present invention include administering to a mammal a combination treatment of a neurotoxin and one or more other agents that may interfere with neurotransmission, neuromuscular transmission, neuronal growth, and/or axonal growth (e.g., sprouting). A combination treatment may result in synergy between two or more compounds such that lower doses of individual compounds are required. A combination treatment may involve the simultaneous or sequential administration of two or more compounds. Therefore, according to the present invention, a neurotoxin may be administered with one or more neuron growth inhibitor simultaneously, or the neurotoxin may be administered prior to the administration of the neuron growth inhibitor, or the neurotoxin may be administered after the administration of a neuron growth inhibitor. In preferred embodiments, the neurotoxin is administered prior to the administration of the neuron growth inhibitor.

Administration of either or both the neurotoxin and the neuron growth inhibitor may be systemic or local. In preferred embodiments, either or both the neurotoxin and the neuron growth inhibitor are administered locally. Examples of localized administrations include, but are not limited to, topical, subcutaneous, subdermal, intramuscular, intracranial, vaginal, optical, anal, pulmonary, and transdermal adminsterations. In preferred embodiments, the administration of the compounds herein is made by topical, subcutaneous, subdermal, or transdermal adminteration. More preferably, administration of compounds herein is made by intramadcular or transdermal microinjections. However, needleless injections are also contemplated.

While preferred embodiments of the present invention have been shown and are described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for treating a condition in a mammal comprising the step of administering to said patient a neurotoxin and a neuron growth inhibitor.

2. The method of claim 1 wherein said administering step results in the inhibition of neurotransmission of a neurotransmitter.

3. The method of claim 2 wherein said inhibition is temporary.

4. The method of claim 2 wherein said inhibition lasts for at least 6 months.

5. The method of claim 2 wherein said neurotransmission is of neurotransmitter acetylcholine.

6. The method of claim 1 wherein the neurotoxin is selected from the group consisting of botulinum toxin, tetanus toxin, curare, bungarotoxin, saxitoxin, and tetrodotoxin.

7. The method of claim 6 wherein the neurotoxin is a botulinum toxin.

8. The method of claim 7 wherein the botulinum toxin is selected from the group consisting of botulinum toxin type A, B, C, D, E, F, and G.

9. The method of claim 8 wherein the botulinum toxin is botulinum toxin type A.

10. The method of claim 1 wherein the neuron growth inhibitor is selected from the group consisting of a Trk receptor inhibitor, a Ras inhibitor, a Raf inhibitor, a Rap-1 inhibitor, a MEK inhibitor, an ERK inhibitor, a PKA inhibitor, a PKC inhibitor, a p53 inhibitor, and a growth factor inhibitor.

11. The method of claim 1 wherein the neuron growth inhibitor is a MEK inhibitor.

12. The method of claim 11 wherein the MEK inhibitor is selected from the group consisting of PD98059, U0126, PD 184352, 2-Cholor-3-(N-succinimidyl)-1,4-naphthoquinone, PD 184352 ARRY-142886, tricyclic flavone, and 2-(2-amino-3-methoxyphenyl)-4-oxo-4H-[1]benzopyran.

13. The method of claim 1 wherein the neuron growth inhibitor is a b-Raf kinase inhibitor.

14. The method of claim 13 herein the neuron growth inhibitor is a b-Raf kinase inhibitor is Rheb, BAY-43-9006, or a Raf kinase inhibitor protein.

15. The method of claim 1 wherein the neurotoxin is administered prior to administration of the neuron growth inhibitor.

16. The method of claim 1 wherein either the neurotoxin or the neuron growth inhibitor is administered locally.

17. The method of claim 1 wherein said condition is selected from the group consisting of a localized dystonia.

18. The method of claim 17 wherein said localized dystonia is selected from the group consisting of cervical dystonia, embouchure dystonia, oromandibular dystonia, spasmodic dystonia, and writer's cramp.

19. The method of claim 1 wherein said condition is a thyroid condition.

20. The method of claim 19 wherein said thyroid condition is selected from the group consisting of hyperthyroidism, hypothyroidism, Graves' disease, goiter, thyroiditis, cancer, and all other conditions that may result in hypothyroidism or hyperthyroidism.

21. The method of claim 1 wherein said condition is a neurological disorder.

22. The method of claim 21 wherein said neurological disorder is selected from the group consisting of a migraine headache, chronic pain (e.g., chronic low back pain), chronic muscle pain (e.g., fibromyalgia), stroke, traumatic brain injury, localized pain (e.g., vulvodynia), cerebral palsy, meige syndrome, hyperhydrosis, tremor, achalasia, secondary and inherent dystonias, Parkinson's disease, spinal cord injury, multiple sclerosis, and spasm reflex.

23. The method of claim 1 wherein said condition is a muscle injury.

24. The method of claim 23 wherein said muscle injury is selected from the group consisting of contusions (bruises), lacerations, ischemia, strains, and complete ruptures.

25. The method of claim 1 wherein said condition is a urological condition.

26. The method of claim 25 wherein said urological condition is selected from the group consisting of pelvic pain, pelvic myofiscial elements, urinary incontinence, prostate disorders, recurrent infection, and urinary retention and bladder dysfunctions.

27. The method of claim 1 wherein said condition is an optical condition.

28. The method of claim 27 wherein said optical condition is selected from the group consisting of blepharospasm, strabismus, and Duane's syndrome.

29. The method of claim 1 wherein said condition is a dermatological condition.

30. The method of claim 29 wherein said dermalogical condition is selected from the group consisting of the appearance of aging skin, wrinkles, eczema, psoriasis, dermatitis, melonoma, pityriasis, and skin cancer.

31. The method of claim 1 wherein said condition is characterized by snoring.

32. The method of claim 1 wherein said condition is a wound.

33. The method of claim 16 wherein said local administration is selected from the group consisting of topically, subdermally, intramuscularly, and subcutaneously.

34. The method of claim 1 wherein said neurotoxin is botulinum toxin type A and is administered at a dose of 0.25-50 units at about every 3 months.

35. A composition for treating or preventing a condition in a patient comprising a neurotoxin and a neuron growth inhibitor.

36. The composition of claim 35 wherein the neuron growth inhibitor is selected from the group consisting of a Trk receptor inhibitor, a Ras inhibitor, a Raf inhibitor, a Rap-1 inhibitor, a MEK inhibitor, an ERK inhibitor, a PKA inhibitor, a PKC inhibitor, a p53 inhibitor, and a growth factor inhibitor.

37. The composition of claim 35 wherein the neuron growth inhibitor is a MEK inhibitor selected from the group consisting of PD98059, U0126, PD 184352, 2-Cholor-3-(N-succinimidyl)-1,4-naphthoquinone, PD 184352 ARRY-142886, tricyclic flavone, and 2-(2-amino-3-methoxyphenyl)-4-oxo-4H-[1]benzopyran.

38. The composition of claim 35 wherein the neuron growth inhibitor is a b-Raf inhibitor selected from the group consisting of Rheb, BAY-43-9006, and a Raf kinase inhibitor protein.

39. The composition of claim 35 wherein the neurotoxin is selected from the group consisting of botulinum toxin, tetanus toxin, curare, bungarotoxin, saxitoxin, and tetrodotoxin.

40. The composition of claim 35 wherein the neurotoxin is botulinum toxin type A.
